SEQUENCE LISTING (18 sequences)

SEQ ID NO: 1 - protein, 7 aa, Homo sapiens
PRKLYDY

SEQ ID NO: 2 - protein, 12 aa, Homo sapiens
LLPDVETPSEED

SEQ ID NO: 3 - DNA, 17 bp, Artificial Sequence
  primer
ctgcttccagatagaga

SEQ ID NO: 4 - DNA, 23 bp, Artificial Sequence
  primer
ttattaggccgcacactgaggga

SEQ ID NO: 5 - protein, 13 aa, Homo sapiens
VLLPDVETPSEED

SEQ ID NO: 6 - protein, 20 aa, Homo sapiens
MFGNGKGYRGKRATTVTGTP

SEQ ID NO: 7 - protein, 28 aa, Homo sapiens
QDWAAQEPHRHSIFTPETNPRAGLEKNY

SEQ ID NO: 8 - protein, 11 aa, Homo sapiens
RNPDGDVGGPW

SEQ ID NO: 9 - protein, 11 aa, Homo sapiens
YTTNPRKLYDY

SEQ ID NO: 10 - protein, 4 aa, Homo sapiens
DVPQ

SEQ ID NO: 11 - protein, 6 aa, Homo sapiens
RKLYDY

SEQ ID NO: 12 - protein, 5 aa, Homo sapiens
KLYDY

SEQ ID NO: 13 - protein, 4 aa, Homo sapiens
LYDY

SEQ ID NO: 14 - protein, 4 aa, Homo sapiens
KLYD

SEQ ID NO: 15 - DNA, 21 bp, Artificial Sequence
  siRNA primer
aacggccgcguggagaucauc

SEQ ID NO: 16 - DNA, 21 bp, Artificial Sequence
  siRNA primer
aagcuguagcguauggugcug

SEQ ID NO: 17 - DNA, 21 bp, Artificial Sequence
  siRNA primer
aagaucacaaucaccaaugac

SEQ ID NO: 18 - DNA, 21 bp, Artificial Sequence
  siRNA primer
aaaucauagcguauggugcug

(19) United States
(12) Patent Application Publication
(10) Pub. No.: US 2005/0053993 A1
(43) Pub. Date: Mar. 10, 2005

(54) Uses of an endothelial cell receptor

(76) Inventor: Donald J. Davidson, Gurnee, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 10/946,789

(22) Filed: Sep. 22, 2004

Related U.S. Application Data
(63) Continuation-in-part of application No. 10/322,853, filed on Dec. 18, 2002.

(51) Int. Cl. C12Q 1/68; C12N 15/85; A61K 31/519; A61K 48/00

(52) U.S. Cl. 435/6; 514/44; 435/455

(57) ABSTRACT

The subject invention relates to uses of a receptor referred to as GRP78 and to other endothelial cell receptors which bind to the kringle 5 region of mammalian plasminogen. More specifically, identification of the functional properties of this receptor and other such receptors allows for the development and screening of agents which, for example, mimic K5 (i.e, mimetics) and therefore inhibit angiogenesis.

### TECHNICAL FIELD

The subject invention relates to uses of a receptor referred to as GRP78 and to other endothelial cell receptors which bind to the kringle 5 region of mammalian plasminogen. More specifically, identification of the functional properties of this receptor and other such receptors allows for the development and screening of agents which, for example, mimic K5 (i.e, mimetics) and therefore inhibit angiogenesis.

### BACKGROUND OF THE INVENTION

Angiogenesis is the process in the body by which new blood vessels are formed. This process is essential for normal body activities including, for example, reproduction, development and wound repair. Under normal biological conditions, angiogenesis is a highly regulated process. However, many diseases are driven by persistent, unregulated angiogenesis.

Several angiogenesis inhibitors are under development or have been developed for use in treating angiogenic diseases (Gasparini et al., J. Clin. Oncol., 13(3):765-782 (1995). Such inhibitors include, for example, suramin and K5. (For a discussion of the properties of K5, see, e.g., Cao et al., Journal of Biol. Chem. 272:22924-22928 (1997), Ji et al., Biochem. and Biophys. Res. Communs. 247:414-419 (1998) and Lu et al., Biochem. and Biophys. Res. Communs. 258:668-673 (1999).) Thus, by analyzing the receptors to which angiogenic inhibitors bind and, in particular, the binding interaction or relationship itself, one may screen for further angiogenic inhibitors as well as purposefully design these inhibitors.

The present inventors have determined that one receptor to which K5 binds is GRP78. This molecular chaperone is constitutively expressed and expression is often dramatically enhanced under stressful conditions such as glucose deprivation, treatment with Ca2+ ionophores, blockage of glycosylation, oxidative stress and hypoxia (Song et al., Cancer Research 61:8322-8330 (2001)). GRP78, also referred to as the immunoglobulin heavy chain binding protein BIP, also plays a role in protecting tumor cells against cytotoxic T lymphocyte-mediated toxicity and the toxic effects of tumor necrosis factor in vitro (Jamora et al., PNAS 93:7690-7694 (1996); see also Lee, A., TRENDS in Biochemical Sciences, Vol. 26, No. 8, pps. 504-510 (2001)). Accordingly, compounds that inhibit or prevent activation of GRP78 may be useful in inhibiting tumor cell growth and/or inducing apoptosis, particularly of hypoxic tumor cells.

In view of the characteristics of the GRP78 protein, as noted above, and the fact that K5 binds to this protein, there is an essential need for other agents, similar to K5, which can bind thereto. Such agents may be used to inhibit angiogenesis as well as other functions of the GRP78 receptor.

All U.S. patents and publications referred to herein are hereby are incorporated in their entirety by reference.

### SUMMARY OF THE INVENTION

The present invention encompasses a method of identifying a composition which inhibits activation of an endothelial cell receptor. The method comprises constructing a vector comprising a nucleotide sequence encoding the endothelial cell receptor and a nucleotide sequence encoding a reporter molecule. The nucleotide sequence encoding the reporter molecule is operably linked to the nucleotide sequence encoding the endothelial cell receptor, introducing the vector into a host cell for a time and under conditions suitable for expression of the endothelial cell receptor, exposing the host cell to a composition which may inhibit activation of the endothelial cell receptor and a substrate specific for the reporter molecule, and measuring the signal generated by reaction of said reporter molecule and said substrate in comparison to that produced by a control host cell, a smaller signal by the host cell into which the modified vector was introduced, indicating that the composition will inhibit activation of the endothelial cell receptor. The receptor may be, for example, GRP78. An example of the composition is K5.

A further embodiment of the present invention encompasses a method of identifying a composition which inhibits expression of an endothelial cell receptor comprising the steps of adding an antibody selected from the group consisting of a monoclonal antibody and a polyclonal antibody produced against the endothelial cell receptor to a solid phase, adding known concentrations of the endothelial cell receptor exposed to the test composition, to the solid phase, in order to form a first complex between the antibody and the known concentrations of the endothelial cell receptor, adding a second antibody to the first complex, selected from the group consisting of a monoclonal antibody and a polyclonal antibody produced against the endothelial cell receptor for a time and under conditions sufficient for formation of a second complex between the first complex and the second antibody, contacting the second complex with an indicator reagent which comprises a signal-generating compound attached to an antibody against the antibody of the second complex, for a time and under conditions sufficient for formation of a third complex, and detecting the presence of a measurable signal, absence of the signal indicating the composition inhibits expression of the endothelial cell receptor and presence of the signal indicating the composition does not inhibit expression of the endothelial cell receptor. The endothelial cell receptor is, for example, GRP78. The composition which inhibits expression of the receptor may be, for example, K5.

A further embodiment of the present invention includes a method of identifying a composition which binds to the GRP78 receptor comprising the steps of exposing the receptor to said composition for a time and under conditions sufficient for formation of a complex and determining presence or absence of said complex, presence of the complex indicating a composition which binds to the receptor. The composition may be attached to an indicator molecule capable of generating a detectable signal. The composition which binds to the GRP78 receptor may be, for example, K5 or a functional equivalent thereof.

Additionally, the present invention includes a method of preventing or treating angiogenesis in a patient in need of such prevention or treatment comprising the step of administering an amount of a composition which binds to at least one endothelial cell receptor sufficient to effect the prevention or treatment. The endothelial cell receptor may be, for example, GRP78, and the composition may be, for example, K5.

Additionally, the present invention includes a method of inducing apoptosis in a tumor cell comprising the step of administering to the cell an amount of a composition which binds to a GRP78 cell receptor on the tumor cell sufficient to effect the induction. Preferably, the composition is a K5. Preferably, the tumor cell is in a state of hypoxia or deficient oxygen supply.

Additionally, the present invention includes a method of inhibiting tumor growth in a patient in need thereof, comprising the step of administering to the patient an amount of a composition which binds to a GRP78 receptor on a tumor cell sufficient to effect the inhibition. Preferably, the composition is a K5. Preferably, the tumor cell is in a state of hypoxia or deficient oxygen supply

### BRIEF DESCRIPTION OF THE DRAWINGS

FIG. 1 illustrates inhibition of I¹²⁵dog-K5 binding to EAHY cells by a polyclonal antibody to GRP78.

FIG. 2 illustrates inhibition of I¹²⁵dog-K5 binding to EAHY cells by various polyclonal antibodies.

FIG. 3 illustrates inhibition of rK5 activity on migration of HMVEC cells with α-GRP78.

FIG. 4 the percent inhibition of proliferation when HUAVEC cells were incubated with rK5 and various concentrations of GRP78 antibody.

FIG. 5 represents avidin-HRP blots of biotinylated cell surface proteins isolated by affinity purification with agarose-K5.

FIG. 6 illustrates GRP78 in EAHY cells starved and then fed at various intervals.

FIG. 7 a determination of the amount of GRP78 present in HMVEC cells starved, exposed to VEGF and stained with a goat polyclonal GRP78 antibody and an anti-goat HRP antibody.

FIG. 8 illustrates the direct binding of recombinant kringle 5 (rK5) with the GRP78 receptor. FIG. 9A is a graph showing inhibition by rK5 of endothelial cell migration induced by a variety of angiogenesis inducers including aFGF, bFGF, IL-8, PDGF, TGF-β, VEGF, HGF and PDGF at concentrations of 50 ng/mL, 15 ng/mL, 40 ng/mL, 1 pg/mL, 100 pg/mL 40 ng/mL and 250 ng/mL respectively. The concentration of rK5 used was 400 pM except for VEGF (rK5=100 pM), PDGF (rK5=100 pM) and HGF (rK5=10 pM). Membranes were covered, fixed and stained and the number of cells that had migrated to the upper chamber per 10 high power field counted. Background migration to DME+0.1% BSA was subtracted and the data reported as the number of cells migrated per 10 high power fields (100×). The results shown are from triplicate experiments (*p<0.01)).

FIG. 9B is a graph showing the induction by rK5 of apoptosis in stimulated endothelial cells. The rate of apoptosis was measured in HMVEC cells using a histone detection kit (Markwell, M. A. K. Anal Biochem 1982, 125, 427-432.) HMVECs were grown in 96 well plates. Recombinant K5 at various concentrations was added to plates and incubated overnight. Apoptosis was determined from triplicate samples and the Apoptotic index was determined by dividing the absorbance from the treated cells by the absorbance from the untreated cells.

FIG. 10A is a graph showing the amount of surface expressed GRP78 on endothelial cells compared to rK5 binding. For all the assays in this figure, 50,000 cells were attached to 96 well plates. Human rK5 potency is highly dependent on the extent of iodination because it contains a readily iodinated tyrosine in its binding sequence. Attempted mutations of this tyrosine to phenylalanine did not allow correct protein folding. However dog K5 has a phenylalanine in this position naturally and is folded and active in these assays. Accordingly ¹²⁵IrK5 (dog) was used as a reagent. ¹²⁵IrK5 (dog) was added to the wells and incubated at room temperature for 1 hour. After 1 hour the cells were washed and the amount of I¹²⁵IrK5 (dog) bound was counted. Scatchard plot analysis shows ¹²⁵IrK5 (dog) bound with a Kd of 0.8 nM with approximately 32,500 binding sites per cell.

FIG. 10B is a graph showing the effect of a K5 active site peptide PRKLYDY (SEQ ID NO:1) and an inactive rK5 N-terminal peptide, LLPDVETPSEED (SEQ ID NO:2) on the binding of ³HK5 (concentration 2 nM) to endothelial cells. Each K5 peptide or a N-terminal antibody to GRP78 was added to HMVECs at various concentrations for 1 hour at room temperature. Cells were washed and the amount of ³HK5 bound was counted.

FIG. 11 is a graph comparing the binding of rK5 to starved, quiescent endothelial cells with that of endothelial cells stimulated by VEGF/bFGF. Tritium-labeled rK5 was added to monolayers of 50,000 HMVEC cells that were either starved or stimulated for 16 hours by 15 ng/mL bFGF and 5 ng/mL VEGF in 96 well plates. The number of counts remaining bound to the cells after extensive washing determined total amount of rK5 bound.

FIG. 12A is a graph showing the effect of an N-terminal GRP78 polyclonal antibody on rK5 inhibition of stimulated endothelial cell proliferation. HMVEC cellular proliferation assay was performed with 10,000 cells added to each well of a 96 well plate. After the cells had attached, complete media containing VEGF (5 ng/mL) and bFGF (15 ng/mL) along with rK5, plus or minus the GRP78 antibody, also was added. The cells were grown for 72 hrs. MTS assay was used to determine the number of live cells. Each point was determined from triplicate samples.

FIG. 12B is a graph showing the effect of N-terminal GRP78 antibody on rK5-induced inhibition of endothelial cell migration. Stimulated HMVEC cell chemotaxis towards VEGF (5 ng/mL) was performed in 96 well plates for 4 hours at room temperature. Casein-AM pre-labeled HMVEC cells that had migrated to the bottom of the membrane were measured by fluorescence.

FIG. 12C is a graph showing the effect of N-terminal GRP78 antibody on rK5-induced apoptosis of endothelial cells. The rate of apoptosis was measured in HMVEC cells using a histone detection kit. HMVECs were grown in 96 well plates. rK5 and an antibody to GRP78 were added to plates and incubated overnight. Apoptosis was determined from triplicate samples and the Apoptotic index was determined by dividing the absorbance from the treated cells by the absorbance from the untreated cells.

FIG. 13 is a graph comparing the effect of binding of rK5 to GRP78 siRNA transfected HMVEC cells and scrambled siRNA transfected cells. GRP78 siRNA or scrambled siRNA transfected EaHy cells were grown in 96 well plates. The media was changed and ³HK5 in PBS was added to the cells. The cells were incubated at room temperature for two hours then washed thoroughly. Cell counts were measure for bound ³HK5. Data points are the average of triplicate studies.

FIG. 14A is a graph showing equilibrium dialysis of recombinant GRP78 (rGRP78) and ³HK5. ³HK5 was added to one side of an equilibrium chamber at concentrations from 0.1 to 3 nM. 2 nM GRP78 was added to the other side of the chamber with a 50,000-kDa mwco filter between. ³HK5 was allowed to equilibrate to both side of the chamber for 72 hours at room temperature. The amount of ³HK5 on the GRP78 side of the chamber was compared to chamber with ³HK5 alone to obtain the bound verses free rK5 distribution.

FIG. 14B is a graph showing competition binding dialysis rGRP78 and ³HK5. Experiments were performed with 2 nM GRP78 and 2 nM ³HK5 using rK5(K82A) mutant and unlabeled rK5 at various concentrations from 0.5 to 50 nM. The amount of ³HK5 on both sides of the chamber was measured after 72 hours and compared to the control.

FIG. 15A is a graph showing the effect of rK5 on HT1080 human fibrosarcoma cells under hypoxic and non-hypoxic conditions. D54 human glioma cells were plated on slides overnight with complete medium. Control slides were put in the incubator at 5% CO₂ and 37° C. The other slide of cells was put in a hypoxic chamber with 95% N₂, 5% CO₂ at 37° C. for 24 hours. GRP78 on the cell surface was detected by IHC analysis with a polyclonal antibody to GRP78 conjugated to horseradish peroxidase. The GRP78 was visualized with the DAB reagent displaying a brown color.

FIG. 15B is a graph showing the effect of rK5 on induction of apoptosis in various tumor cell lines tested under hypoxic conditions. Cells were plated in 96-well plates overnight. Half the plates were put in hypoxic chambers overnight at 37° C. with and without 500 nM rK5. Increased apoptosis was shown by an ELISA technique that measures the number of nucleosome fragments. The apoptotic index was calculated from the apoptosis rate of control cells (without K5 and with or without hypoxia). The samples were run in quadruplicate.

FIG. 15C is a graph showing the effect of rK5 on hypoxia-stressed HT1090 cells transfected with an siRNA and a scrambled siRNA to GRP78. The transfected cells were plated in 96 well plates and placed at 5% CO₂ and 37° C. rK5 (500 nM) was added to the cells and half were incubated in an hypoxic chamber with 95% N₂, 5% CO₂ at 37° C. and half were incubated at 5% CO₂ and 37° C. for 24 hours. Apoptosis was shown by an ELISA technique mentioned above. The samples were run in quadruplicate.

FIG. 15D is a graph showing the effect of rK5 on cell growth of Adriamycin-treated D54 cells. Adriamycin (50 nM) was added to 96-well plates containing 50,000 D54 human glioma cells. rK5 was added to the cells with and without Adriamycin (5% CO₂ at 37° C. for 72 hours). The number of cells was then determined by an MTS assay. Each data point is an average of three experiments.

FIG. 16 is a graph showing the effect of rK5 and hypoxia treatment on caspace 7 activity in HT1080 cells. HT1080 cells were plated in 48 well plates. rK5 at 100 nM was added to the plates. HT1080 cells were incubated overnight in hypoxic conditions (95% N₂, 5% CO₂) compared to controls, which were incubated in 5% CO₂. Caspase 7 activity in cell lysates was detected by the activation of an ELISA fluorescent substrate (R&D Systems).

### DETAILED DESCRIPTION OF THE INVENTION

As noted above, the present inventors have discovered that K5 and, in particular, the active site (PRKLYDY) thereof, binds to the endothelial cellular receptor GRP78. Based upon this finding, this protein may be utilized for many purposes. For example, the protein may be used to screen for and identify analogs or mimetics of K5 which bind to the protein also and should therefore be functional equivalents of K5. Such analogs or mimetics may inhibit or suppress angiogenesis in a patient. One may also screen for antagonists and allosteric modulators of the receptor, thereby also reducing or preventing angiogenesis in the patient. One may also screen for agonists of the receptor. (For purposes of the present invention, a “functional equivalent” is defined as a compound or entity which behaves in the same manner, in terms of binding, as the entity to which it is being compared.) Additionally, one may use the receptor to identify compositions that inhibit expression of the receptor. Moreover, the protein may be used in order to further comprehend the binding properties of K5 to a receptor on the cell surface.

Once a useful pharmaceutical composition is identified, it may comprise a therapeutically effective amount of the inhibitor or modulator and an appropriate physiologically acceptable carrier (e.g., water, buffered water or saline). The dosage, form (e.g., suspension, tablet, capsule, etc.), and route of administration of the pharmaceutical composition (e.g., oral, topical, intravenous, subcutaneous, etc.) may be readily determined by a medical practitioner and may depend upon such factors as, for example, the patient's age, weight, immune status, and overall health.

Another embodiment of the present invention encompasses a method of assaying test samples (e.g., biological fluids) for the presence or absence of the GRP78 receptor. Thus, for example, a patient having a malignancy may be tested for presence of the receptor based upon the binding assays described herein.

The drug screening assays referred to above will now be described in detail. For example, in one method, a vector is created comprising an isolated DNA sequence encoding the GPR78 receptor. This sequence may be attached to, for example, a nucleotide sequence encoding a reporter molecule (e.g., an enzyme such beta-galactosidase) or entity capable of interacting with a substrate, thereby emitting or generating a measurable signal. The vector may be, for example, a plasmid, a bacteriophage or a cosmid. The vector is then introduced into host cells under time and conditions suitable for expression of the receptor. (The host cells may be prokaryotic or eukaryotic cells.) The host cells are then exposed to the test composition thought to, for example, inhibit activation of the receptor. The cells are also exposed to the relevant substrate. One then measures the quantity of signals emitted from the reporter molecule-substrate reaction. If the amount of signals produced by the host cells, exposed to the composition in question, is lower than that produced by control cells (i.e., cells which have not been exposed to the composition), then the composition has inhibited the activity of the receptor. If the amount of signal produced by the treated cells is equal to that produced by the control cells, the composition has not inhibited the activity of the receptor. (See, e.g., U.S. Pat. Nos. 5,912,122, 5,912,120 and 5,919,450.)

Additionally, the present invention covers an Affinity-Selection method, using purified receptor in a filtration assay, to identify compositions that bind to the receptor to prevent the receptor from binding to other agents, interacting with agents, etc., thus preventing the receptor from functioning as it would normally in vivo. Briefly, purified receptor is mixed with several test compounds. The mixture is passed through a filter which only allows certain molecular weight molecules to pass through. Compositions that bind to the receptor will be retained by the filter. The unbound compounds are not retained and can be separated from the bound compositions. The structures of the compositions which bind to the receptor are determined, for example, by Mass Spectrometry.

Furthermore, the present invention also encompasses a receptor binding method using radiolabeled receptor to bind to cells or membranes prepared from tissues or cells containing GRP78 receptors. In this manner, one may identify compositions that block GRP78 from binding to agents to which it would normally bind, thus preventing the receptor from functioning. In particular, the purified recombinant receptor protein from, for example, mammalian cells is radiolabeled ([¹²⁵I], [³H], [¹⁴C], etc.). The radiolabeled receptor is then incubated with cells or membranes prepared from tissues or cells which contain the GRP78 receptors in the presence or absence of the test composition. Radiolabeled cells and membranes are then separated from non-radiolabeled cells and membranes by separation methods such as, for example, filtration and centrifugation. The amount of receptor binding to cells or membranes is determined by counting radioactivity. A decrease in radioactivity in the presence of a test composition indicates that the composition inhibits receptor binding, and thus is useful in inhibiting receptor function.

The present invention also covers two methods, using which identify compositions that inhibit the synthesis and expression of the receptor. In the sandwich method, a mammalian monoclonal and/or polyclonal antibody (e.g., rabbit or mouse) against the mature form of the receptor is coated on a solid surface (e.g., the Immulon-4 plate (Dynatech Laboratories Inc., Chantilly, Va.)). The surface will be blotted by a known blotting agent, for example, Bovine Serum Albumin (BSA), and washed. Samples or known concentrations of purified GRP78 are added to the surface (e.g., plate). After the receptor binds to the antibody or antibodies, the surface will be washed, and then incubated with a mammalian monoclonal and/or polyclonal antibody (e.g., goat, rabbit or mouse) raised against the receptor. The binding of the second anti-receptor antibody will be detected by use of an indicator reagent which comprises an antibody conjugated with a signal-generating compound, for example, an enzyme. A substrate for the enzyme is also added if an enzyme is utilized. For example, horseradish peroxidase (HRP) and its substrate O-Phenylenediamine hydrochloride (OPD) may be utilized. In particular, the enzyme-substrate reaction generates a detectable signal or change, for example, color, which may be read, for example, in a Microplate Reader. Examples of signal generating compounds, other than an enzyme which may be utilized include, for example, a luminescent compound, a radioactive element, a visual label and a chemiluminescent compound. Known concentrations of the receptor are used to generate a standard curve. The concentration of receptor in the unknown samples can be determined using the standard curve. The test agents that decrease the receptor concentration in supernatants are potentially useful for inhibition of receptor synthesis on the endothelial cell.

In the competitive method, a fixed amount of the receptor is coated on a solid surface, for example, the Immulon-4 plate. The plate will be blotted by, for example, BSA or another known blotting agent, and washed. Samples are added to the plate along with a mammalian monoclonal and/or polyclonal antibody (e.g., goat, rabbit or mouse) against the receptor. The plate is washed, and then incubated with an indicator reagent comprising an antibody conjugated with a signal-generating compound, for example, an enzyme (or the entities described above). If an enzyme is used, a substrate for the enzyme is also provided. The enzyme may be, for example, horseradish peroxidase (HRP) The substrate may therefore be O-Phenylenediamine hydrochloride (OPD)). Again, the enzyme-substrate reaction generates a detectable change or signal, for example, color, which can be read in, for example, a microplate reader. Known concentrations of purified receptor may be used to generate a standard curve. The concentration of receptor in the unknown samples can be determined using the standard curve. The test agents which decrease the receptor concentration in supernatants are potentially useful for inhibition of receptor synthesis by the cell. Known concentrations of the receptor, or receptor in the sample, compete with receptor protein coated on the plate in binding to receptor antibodies. When more receptor is present in the sample, a smaller signal is generated. If a test agent is able to block receptor, the amount of receptor in that particular sample will be less than in the control, and the signal in that sample will be more than in the control.

The present invention may be illustrated by the use of the following non-limiting examples:

### Materials and Methods

Materials. Recombinant basic fibroblastic growth factor (bFGF) and vascular endothelial growth factor (VEGF) were obtained from Invitrogen (San Diego, Calif.). Polyclonal C-terminal (C-20) and N-terminal (N-20) GRP78 antibodies were obtained from Santa Cruz, Inc. (Santa Cruz, Calif.). A K5 monoclonal antibody was obtained from Green Mountain Antibodies (Burlington, Vt.). All other antibodies used were obtained from Cell Signaling Technology, Inc (Beverly, Mass.).

Cell Culture. Human Microvascular Endothelial Cells-Dermal (HMVEC), Human Umbilical Arterial Vascular Endothelial Cells (HUAVEC), Human Umbilical Endothelial Cells (HUVEC), Dermal Fibroblast and Neutrophils were obtained from Clonetics Corporation (San Diego, Calif.). D54 human glioma tumor cells were obtained from University of Texas-Southwestern Medical Center (Houston, Tex.). All other cell lines were obtained from American Type Culture Collection (Manassas, Va.). Peptide synthesis. All peptides were synthesized using a Symphony (Protein Technology Inc., Woburn, Mass.) automated peptide synthesizer. Peptide purification was performed using a Gilson HPLC system equipped with automated liquid handler. The Fmoc-protected amino acids and resins were purchased either from Calbiochem-Novabiochem Corp., (San Diego, Calif.) or from Bachem Inc. (Torrance, Calif.). Mass spectra were recorded using either a Finnigan SSQ7000 (ESI) or JEOL JMS-SX102A-Hybrid (FAB) mass spectrometers.

Cell Proliferation Assay. The effect of rK5 and rK5 peptides on endothelial cells were assessed using a proliferation assay with 1% BSA and 3 ng/ml bFGF in serum-free media. Relative cell numbers in each well of a 96 well microplate after incubation for 72 hours in the absence or presence of inhibitors were determined by using the AQueous cell proliferation assay (Promega, San Luis Obispo, Calif.). For all other cell lines tested for proliferation, minimal growth media was used (Cao, Y., Ji, R., Davidson, D., Schaller, J., Marti, D., Sohndel, S., McCance, S., O'Reilly, M., Llinas, M., and Folkman, J. Kringle domains of human angiostatin. Characterization of the anti-proliferative activity on endothelial cells. (1996) J. Biol. Chem. 271, 29461-29467). Results are presented as the percent inhibition of control cell (bFGF-induced) proliferation.

Expression and purification of rK5. Kringle 5 fragment was PCR amplified from a human plasminogen cDNA template (American Type Culture Collection, Manassas, Va.) with the following two primers: 5′-CTGCTTCCAGATAGAGA-3′ (forward primer for residue 450-457, SEQ ID NO:3) and 5′-TTATTAGGCCGCACACTGAGGGA-3′ (reverse primer for plasminogen residues 538-543, SEQ ID NO:4). The PCR fragment was ligated into the pET32a vector (Novagen, San Diego, Calif.) that had been digested with NcoI and XhoI The NcoI and XhoI cleavage sites of the pET32a had been filled in to form blunt ends with pfu DNA polymerase (2.5 units/μl, Stratagene, La Jolla, Calif.). XL2-Blue Ultracompetent cells (Stratagene) were transformed with the ligation mixture as per the manufacturer's instructions. After sequence confirmation, the pET32a/K5 vector was retransformed into E. coli BL21 cells (DE3) (Novagen) for expression as per the manufacturer's instructions. The recombinant protein was recovered from the cell paste by cell lysis in lysis buffer (50 mM Tris/300 mM NaCl/1 mM MgCl₂, pH 7.8) using a french press. The His-tagged protein was purified over a Probond nickel resin (Invitrogen). The His-Tag was removed from the rK5 molecule by enterokinase (Invitrogen) and the rK5 was re-purified over a second Probond nickel column to remove the His-tag. Finally, endotoxin contamination was removed by size filtration (5 kDa) chromatography.

Yeast rK5 was expressed as previously described (Chang, Y, Mochalkin, I, McCance, SG, Cheng, B, Tulinsky, A, Castellino, FJ Structure and Ligand Binding Determinants of the Recombinant Kringle 5 Domain of Human Plasminogen. Biochemistry 1998, 37, 3258-3271). Briefly, the human K5 gene was expressed in the methylotrophic yeast Pichia patoris (Invitrogen). Genetic transcription of rK5 was under the control of the alcohol oxidase promoter (AOX1). The AOX1 promoter permits high-level expression of heterologous proteins in Pichia. The K5 expression construct also includes a secretion signal sequence to direct transport of the protein to the medium. The plasmid construct was a hybrid of commercially available plasmid sequences from Invitrogen, designated pHIL-S1 and pHIL-D2. The expressed rK5 was purified by octyl-sepharose and size exclusion chromatography.

Radiolabeled rK5. rK5 was tritiated (3H) by a method previously published (Bush G A, Yoshida N, Lively M O, Mathur BP, Rust M, Moran TF, Powers JC. Ion beam tritium labeling of proteins and peptides. J. Biol. Chem. 1981 Dec. 10;256(23):12213-21) Briefly, a carefully controlled particle beam composed of T3+ and T2+ions and fast T2 molecules were accelerated into rK5 within a vacuum chamber. The ³HK5 was found to be active in the endothelial cell migration assay with an IC50 of 0.2 nM and a specific activity of 8.74 mCi/mg.

Human rK5 potency is highly dependent on the extent of iodination because the molecule contains a readily iodinated tyrosine in its binding sequence. Mutations of this tyrosine to phenylalanine resulted in incorrect protein folding. However dog rK5 has a phenylalanine in this position naturally and is folded and active in our assays. We therefore relied on ¹²⁵IrK5 (dog) as a reagent. The radioiodination of rK5 (dog) was performed following the procedure published by Markwell (Markwell, M. A. K. Anal Biochem 1982, 125, 427-432). The Iodobead reagent (Pierce) was used for the radioiodination, and the labeling reaction as per protocol. A total of two beads were used with 25 μg of rK5 for the reaction. The separation of labeled rK5 from free iodine was accomplished using an iodine trap and a desalting spin filter (Pierce Chemical Co., Racine, Wis.). ¹²⁵¹K5(dog) was found to be activity in the migration assay.

Endothelial cell migration assays: The effect of rK5 on endothelial cell migration was determined by two different methods. The first assay was performed in a 96 well plate with a cellulose membrane between the upper and lower chambers. HMVEC were starved of growth factors overnight, labeled with fluorescent calcein AM (50-100 nM), plated into a 96 well migration chamber (2.9×10⁴/well) (Neuroprobe, Gaitherburg, Md.), and stimulated to migrate with VEGF (5 ng/mL). After 4 h, migrated cells were measured by fluorescence (Frevert C W, Wong V A, Goodman R B, Goodwin R, Martin T R. Rapid fluorescence-based measurement of neutrophil migration in vitro. J Immunol Methods. 1998 Apr. 1;213(1):41-52). In a second assay for cellular migration, a standard Boyden chamber was used (Polyerini, P. J., Bouck, N. P. & Rastinejad, F. Assay and purification of naturally occurring inhibitor of angiogenesis. Meth. Enzymol. 198, 440-450 (1991)). HMVEC cells were starved overnight in DME containing 0.1% bovine serum albumin (BSA) and harvested by scraping and resuspended in DME with 0.1% BSA at 1.5×10⁶ cells per ml. Cells were added to the bottom of a 48-well, Boyden chamber. The chamber was assembled and inverted, and cells were allowed to attach for 2 hours at 37° C. to polycarbonate chemotaxis membranes (5 μm pore size) that had been soaked in 0.1% gelatin overnight and dried. The chamber was re-inverted, test substances, including activators were added to the wells of the upper chamber and the apparatus was incubated for 4 hours at 37° C. Growth factors were used, where indicated, at concentrations determined in preliminary experiments to give equivalent migration responses of about 100 cells migrated/high powered field (400×). Growth factors and concentrations used were aFGF (50 ng/ml), bFGF (15 ng/ml), IL-8 (40 ng/ml), TGFb (1 pg/ml), VEGF (100 pg/ml), HGF (40 ng/ml) and PDGF (250 pg/ml). Membranes were recovered, fixed and stained and the number of cells that had migrated to the upper chamber per 10 high power fields counted. Background migration to DME+0.1% BSA was subtracted and the data reported as the number of cells migrated per 10 high power fields (400×) or, when results from multiple experiments were combined, as the percent inhibition of migration compared to the positive growth factor control (Polyerini, P. J., Bouck, N. P. & Rastinejad, F. Assay and purification of naturally occurring inhibitor of angiogenesis. Meth. Enzymol. 198, 440-450 (1991)).

Assessment of Cellular Apoptosis: The effects of rK5 and rK5 peptide-induced apoptosis were determined with a histone ELISA apoptosis assay (Roche, Indianapolis, Ind.)(Mayr M, Li C, Zou Y, Huemer U, Hu Y, Xu Q. Biomechanical stress-induced apoptosis in vein grafts involves p38 mitogen-activated protein kinases. FASEB J. 2000 Feb.; 14(2):261-70) Cells (5000 per well) were grown in 96 well plates. rK5 and/or an antibody to GRP78 were added to plates and incubated overnight. Apoptosis was determined from triplicate samples and the Apoptotic Index was determined by dividing the absorbance from the treated cells by the absorbance from the untreated cells.

Binding of human ¹²⁵IrK5 to Endothelial Cells: Tritium-labeled rK5 was added to monolayers of 50,000 HMVEC cells that were either starved or stimulated for 16 hours by 15 ng/ml bFGF and 5 ng/ml VEGF in 96 well plates. The number of counts remaining bound to the cells after extensive washing determined total amount of rK5 bound (Dudani A K, Ganz P R. Endothelial cell surface actin serves as a binding site for plasminogen, tissue plasminogen activator and lipoprotein(a). Br J Haematol. 1996 October; 95(1): 168-78). Binding of ¹²⁵IK5 (dog) to Endothelial Cells and rK5 to Recombinant GRP78: The same methods as described above for the expression and purification of human K5 were used to express dog rK5 in E. coli. ¹²⁵IK5 (dog) was added to the wells and incubated at room temperature for 1 hour. After 1 hour the cells were washed and lysed with M-Per (Pierce, Racine, Wis.) and the amount of ¹²⁵IK5 (dog) bound was counted. Scatchard plot analysis was performed using Prism (GraphPad Software, Inc., San Diego, Calif.) software. Competition binding against 5 nM ¹²⁵IK5 (dog) was tested using a monoclonal antibody against GRP78 (N-20, Santa Cruz, Calif.) or a monoclonal antibody against K5 (Green Mountain Antibodies, Burlington, Vt.). The antibodies were added to HMVECs at various concentrations for 1 hour at room temperature. Cells were washed and the amount of bound ¹²⁵IK5 (dog) bound was counted. Competition binding was also tested against 2 nM 3HK5. K5 peptides or a c-terminal antibody to GRP78 (A-129, Santa Cruz, Calif.) were added to HMVECs at various concentrations for 1 hour at room temperature. Cells were washed, lysed and the amount of ³HK5 bound was estimated by scintillation counting.

Immunohistochemical Analysis of GRP78 on HMVEC Cells:

Cells were starved overnight with media alone. Complete media, containing 10% FBS plus 15 ng/ml bFGF and 5 ng/ml VEGF, was added at different times to the cells. GRP78 bound antibody was visualized with horseradish peroxidase (HRP) reactive substrate visualized by a brown color.

Binding and Pull Down of rK5 Binding Proteins. Binding of rK5 to endothelial cells was measured as described (Zhang, J. C., Donate, F., Qi, X. Ziats, N. P., Juarez, J. C., Mazar, A. P., Pang, Y. P., McCrae, K. R. The antiangiogenic activity of cleaved high molecular weight kininogen is mediated through binding to endothelial cell tropomyosin. PNAS 2002 Sep. 17; 99(19):12224-12229). Briefly, HMVEC cells (50,000 cells/well) were cultured in 96 well microtiter plates for 2 hours, washed and then incubated with PBS and increasing concentration of ³HK5 or ¹²⁵¹K5(dog) for another 2 hours at 4° C. After washing, cells were lysed and bound labeled rK5 was counted.

Cell surface rK5 binding proteins were isolated by two methods. The first method used N-terminal biotinylated-PRKLYDY (SEQ ID NO:1) active site rK5 peptide with 5×10⁷ endothelial or tumor cell lysate. Cell lysate was passed over an agarose-avidin-biotin-PRKLYDY (SEQ ID NO:1) column. The column was washed with two column volumes of 100 nM of the N-terminal rK5 peptide. Bound proteins were eluted with excess unlabeled rK5. Mass spectrometry analysis was used to determine the bound proteins.

The second method used to identify cell surface rK5 binding proteins was described previously (Zhang, J. C., et al, supra). Surface proteins on 4×10⁶ EaHy cells were labeled with NHS-biotin. The cells were washed and lysed (M-Per, Pierce). Cell lysates were mixed with S-tag K5 for 1 hour at room temperature. S-tag K5 bound proteins were precipitated with S-protein agarose (Pierce). Bound proteins were eluted with excess rK5 or with excess PRKLYDY (SEQ ID NO:1) peptide. Eluted proteins were visualized with avidin-HRP and a chemiluminescent substrate. Mass spectroscopic analysis was used to identify the major protein bands.

Binding of rK5 to rGRP78 was measured using equilibrium dialysis (Kariv I, Cao H, Oldenburg K R. Development of a high throughput equilibrium dialysis method. J Pharm Sci. 2001 May; 90(5):580-87). In the top well of a 96 well equilibrium dialyzer (molecular weight cut off 50K Daltons, Harvard Apparatus, Holliston, M A) 150 μl of 10 nM rGRP78 was added. In the reciprocal (bottom) chamber, 150 μl of increasing concentrations from 0.1 to 50 nM ³HK5 was added. The chambers were shaken at room temperature for 72 hours. The total number of counts from both chambers after dialysis was compared with the number of counts remaining in the ³HK5 chamber.

RNA interference: RNA interference (RNAi) of GRP78 expression was induced with short interfering RNA (siRNA) directed against the GRP78 mRNA. Three different nucleotide siRNA primers were made that targeted human GRP78 mRNA sequence. The siRNAs started at position 139 (A.A.C G.G.C . C.G.C . G.U.G . G.A.G . A.U.C . A.U.C [SEQ ID NO:15]), position 1175 (A.A.G . C.U.G . U.A.G . C.G.U A.U.G . G.U.G . C.U.G. [SEQ ID NO:16]) and position 1567 (A.A.G . A.U.C . A.C.A . A.U.C . A.C.C . A.A.U . G.A.C [SEQ ID NO:17]). A scrambled, si-RNA from position 1567 was used as negative control (A.A.A . U.C.A . U.A.G . C.G.U A.U.G . G.U.G . C.U.G. [SEQ ID NO:18]). All oligonucleotides were from Dharmacon Research (Dharmacon RNA Technologies, Lafayette, Colo.).

EaHy or HT1080 cells were seeded at a density of 20 000 cells/cm² the day before transfection and were approximately 40% confluent when they were transfected with 50 nM positive or scramble oligonucleotides in Lipofectamine 2000 (Invitrogen) and Optimem (Life Technologies) without serum or BSA. Before transfection, the cells were washed once with Optimem. Transfection medium was maintained on cells for 3 hours, it was then removed and substituted with complete medium. The reduction in GRP78 protein, 48 hours after transfection, was estimated by Western blot analysis (30).

In selected studies, GRP78 siRNA- or scrambled siRNA-transfected EaHy cells were grown in 96 well plates. The media was changed and ³HK5 in PBS was added to the cells at various concentrations. The cells were incubated at room temperature for two hours then washed thoroughly. Cell counts were measured for bound ³HK5 as described above. Data points were calculated from the average of triplicate samples.

### EXAMPLE 1

### Identification of an Endothelial Cell K5 Receptor

The normal function of GRP78 is to chaperone and help fold proteins in the endoplasmic reticulum. Under stressed conditions, unfolded or improperly folded proteins are chaperoned by GRP78 to proteosomes for degradation. Under hypoxic stressed conditions, GRP78 and a close relative to GRP96, HSP90, are found on cell surfaces. Published reports of over expression, antisense, and ribozyme approaches in tissue culture systems suggest that GRP78 can protect cells against cell death. In a variety of cancer cell lines, solid tumors and human biopsies, the level of GRP78 is elevated, correlating with malignancy. In addition, induction of GRP78 has been shown to protect cancer cells from immune surveillance and apoptosis, whereas suppressing the stress-mediated induction of GRP78 enhanced apoptosis, inhibited tumor growth and increased the cytotoxicity of chronic hypoxic cells.

To determine if GRP78 is a cell surface receptor for K5, a goat polyclonal antibody to GRP78 was used to compete with K5's binding to EAHY cells. EAHY cells (20,000 per well) were let adhere to 96 well plates; the cells were then incubated with α-GRP78 and I¹²⁵dog-K5 for 1 hour at 4 C. Media was removed and the cells were washed 5× with cold PBS. Cells were lysed and bound I¹²⁵dog-K5 counted. Assays were run with eight replicates each.

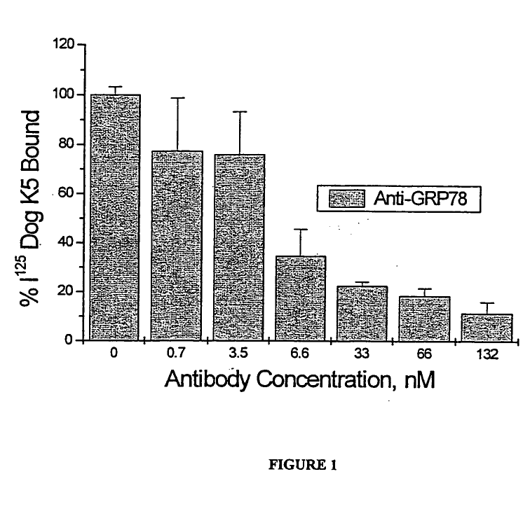

The polyclonal antibody to GRP78 inhibited the binding of 5 nM I¹²⁵K5 (dog) in a dose dependent manner with and IC50 about 6 nM (FIG. 1). As a comparison, EAHY cells (20,000 per well) were let adhere to 96 well plates. The cells were then incubated with the various antibodies and I¹²⁵dog-K5 for 1 hour at 4 C. The media was removed and the cells were washed 5× with cold PBS. Cells were lysed and bound I¹²⁵dog-K5 counted. Assays were run with eight replicates each.

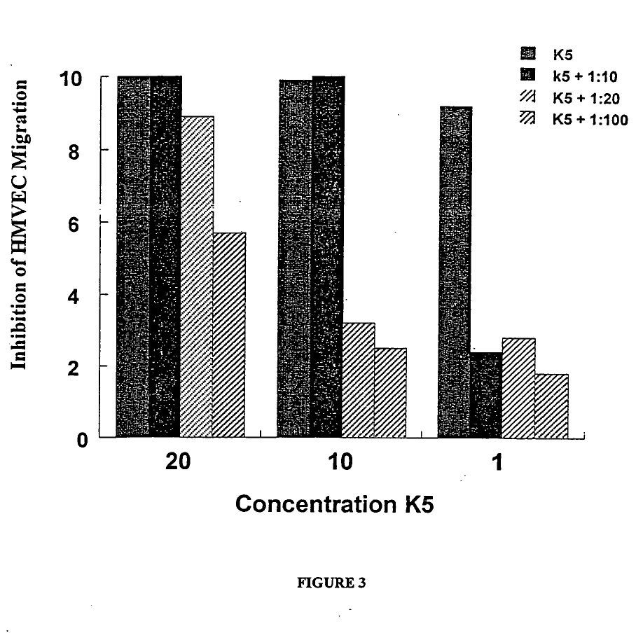

A monoclonal antibody raised against K5 also inhibited I¹²⁵K5 (dog)'s binding to EAHY cells with an IC50 around 15-20 nM, and the panel of various goat polyclonal antibodies weakly inhibited K5's binding to EAHY cells (FIG. 3).

### EXAMPLE 2

### Inhibition of Recombinant K5 Activity on Migration of HMVEC Cells With α-GRP78

Since the antibody to GRP78 could inhibit K5's binding to endothelial cells, it should also inhibit K5's activity on endothelial cell migration and proliferation.

In particular, MVEC cells were labeled with Casein-AM. The cells were loaded on to the top chamber of a 96 well migration plate. The bottom wells were preloaded with media containing VEGF (10 ng/ml), rK5 (100 nM) and various concentrations of α-GRP78. The plates were incubated at 37C for 4 hours. Membranes were removed and the underside was counted with a fluorometer for cell migration. Assays were run in triplicate. The data obtained is shown in FIG. 3.

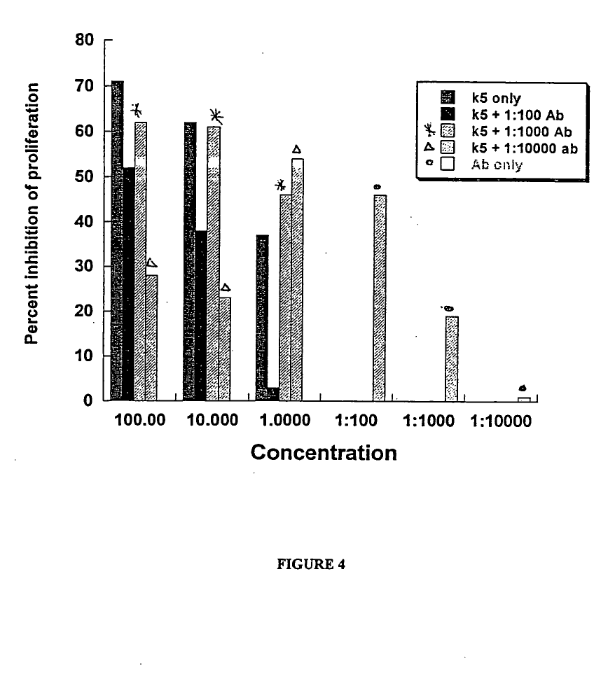

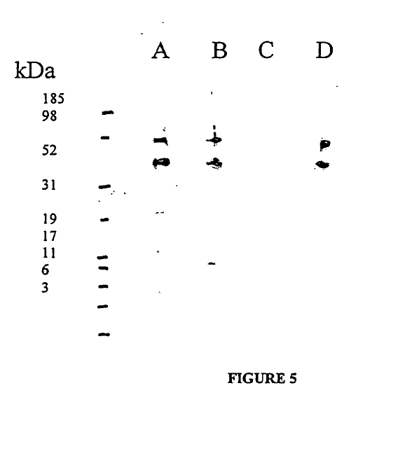

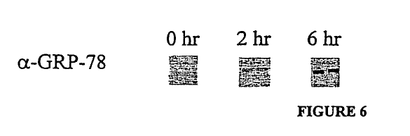

Additionally, HUAVEC cells were incubated with rK5 and various concentrations of GRP78 antibody. The amount of labeled thymidine incorporated was determined after 24 hours and was used to calculate percent inhibition of proliferation compared to untreated cells. The green line displays proliferation inhibition of cells with α-GRP78 alone. As can been seen in FIG. 4, the α-GRP78 at higher concentrations does inhibit cell proliferation, however at lower concentrations (1:10000) this inhibition is not observed. At a 1:10,000 dilution of α-GRP78, the inhibition of K5 activity on endothelial cell proliferation was dose dependent.

In view of the above, in HMVEC migration (FIG. 3) and HUAEC proliferation assays (FIG. 4), anti-GRP78 inhibited rK5's activity in a dose dependent manner.

### EXAMPLE 3

### AVIDIN-HRP Blots of Biotinylated Cell Surface Proteins Isolated by Affinity Purification with AGAROSE-K5

To determine if GRP78 is found on the cell surface of stimulated cells, surface proteins on EAHY cells were labeled with biotin. The cells were then lysed and affinity purification of S-tag-K5 binding proteins was performed.

Avidin-HRP was used to visualize biotinylated (cell surface) proteins. Two major proteins at molecular weights of ˜75 kDa and ˜95 kDa were isolated that contained biotin label (FIG. 5). These two protein's binding to the S-tag K5 column could also be competed with excess rK5 or the K5 active site peptide (PRKLYDY) (FIG. 5, lane B and lane C) whereas the N-terminal peptide of K5 did not inhibit either protein from binding to the S-tag K5 column.

In particular, surface proteins on 1×10⁶ EAHY cells were labeled with NHS-biotin. The cells were washed 3× with PBS and lysed with M-pur. Cell lysates were mixed with (A) 100 nM S-tag-K5, (B) 100 nM S-tag-K5 plus 1 μM PRKLYDY, (C) 100 nM S-tag-K5 plus 10 μM cold rK5 and (D) 100 nM S-tag-K5 and N-terminal K5 peptide at 1 μM for 1 hour at room temperature. The K5 binding proteins were precipitated with S-protein-agarose. Bound proteins were eluted with 50 mM glycine buffer at pH 3.0 and run for PAGE analysis. Surface proteins (biotinylated) that bind K5 were visualized with avidin-HPR and a chemiluminescent substrate.

These results strongly suggest that GRP78 is found on the surface of stimulated endothelial cells and K5 binds to GRP78.

### EXAMPLE 4

### Visualization of GRP78 with a Chemiluminescent Substrate

Since the binding of rK5, to stimulated endothelial cells, is upregulated about 4-10 fold, as compared to starved cells, the level of GRP78 on endothelial cells should also be up regulated.

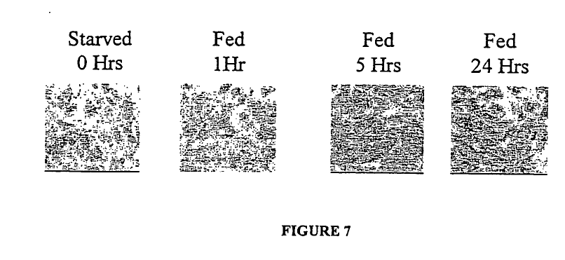

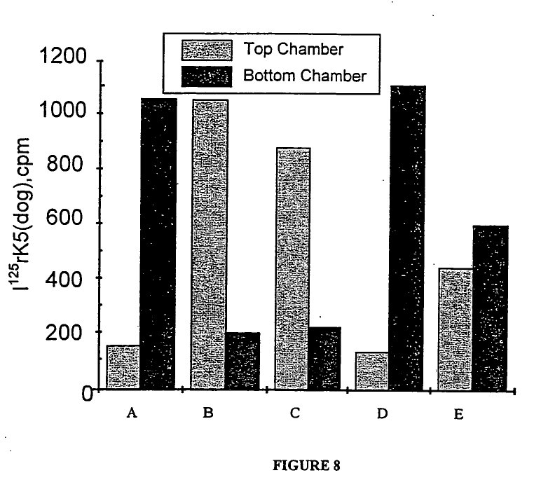

In FIG. 7, the levels of GRP78 protein were analyzed after cells were starved overnight and then fed complete media containing 100 ng/mp VEGF. At times indicated after feeding (see FIG. 7), whole cell lysates were run on PAGE and blotted with a polyclonal GRP-78-HRP antibody. The GRP78 was visualized with a chemiluminescent substrate. Within 4 hours, the cellular protein levels of GRP78 dramatically increase after VEGF stimulation. This is also observed on endothelial cell surfaces by comparison of anti-GRP78 binding on starved as well as VEGF stimulated cells (FIG. 7). HMVEC cells, grown on glass slides, were starved for 26 hours, and then the media was replaced at various times with complete media containing 100 ng/ml VEGF. The cells were washed, fixed and stained for GRP78 with a goat polyclonal GRP78 antibody and an anti-goat HPR antibody. Determination of the amount of GRP78 present was determined by precipitation of a MTB substrate to give a dark brown color.

### EXAMPLE 5

### Direct Binding of Recombinant Kringle 5 with GRP78

Ultra centrifugation with 50,000 MW cut off filters was used with iodinated recombinant dog K5 (I¹²⁵rKS(dog)) and GRP78 (bovine brain). The GRP78 and I¹²⁵rK5 (dog) were incubated for 1 hour at room temperature. The solution was then transferred to ultracentrifuges and spun at 10,000×g for 2 min. The top chamber contained GRP78 (mw 78,000) and I¹²⁵rK5 (dog) that bound. The top and bottom solutions were counted for I125. Column A: 1 nM I¹²⁵rK5(dog). Column B: 1 nM I¹²⁵rK5 (dog)+1.5 nM GRP78. Column C: 1 nM I¹²⁵rK5(dog)+1.5 nM GRP78+100 nM N-terminal K5 peptide, LLPDVETPSEED. Column D: 1 nM I¹²⁵rK5(dog)+1.5 nM GRP78±100 nM active site K5 peptide PRKLYDY. Column E: 1 nM I¹²⁵rK5(dog)+1.5 nM GRP78+1 nM rK5 (unlabeled). The GRP78 bound to the labeled rK5 causing its retention on the top of the filter. This binding can be inhibited by rK5 or the K5 active site peptide but not an inactive K5 N-terminal peptide.

### EXAMPLE 6

### Recombinant K5 (rK5) and K5 Active Site Peptides Inhibit Endothelial Cell Activity

It previously was reported that rK5 has antiangiogenic activity in vitro, inhibiting bovine endothelial cell proliferation with an IC50 value approximately 50 nM (Cao, Y., Ji, R., Davidson, D., Schaller, J., Marti, D., Sohndel, S., McCance, S., O'Reilly, M., Llinas, M., and Folkman, J. Kringle domains of human angiostatin. Characterization of the anti-proliferative activity on endothelial cells. (1996) J. Biol. Chem. 271, 29461-29467). The present experiments expand on those results using a yeast expressed rK5 (no detectable endotoxin) as well as synthetic K5 peptides to examine their effects on stimulated human endothelial cell proliferation, migration and apoptosis assays. These assays show that rK5 inhibits stimulated human endothelial cell migration in a dose-dependent manner with an IC50 value of 0.20 nM (Table 1 below).

TABLE I
Inhibition of VEGF stimulation HMVEC migration^{a}

| | |
|---|---|
| SEQ | IC50 values |

| | | |
|---|---|---|
| Peptides from Kringle 5 | ID NO: | (nM) |

| | | |
|---|---|---|
| Kringle 5 (450-543) | | 0.20 ± 0.01 |
| Ac-VLLPDVETPSEED-NH2 | 5 | >1,000 |
| Ac-MFGNGKGYRGKRATTVTGTP-NH2 | 6 | >1,000 |
| Ac-QDWAAQEPHRHSIFTPETNPRAGLEKNY- | 7 | >1,000 |
| NH2 | | |
| Ac-RNPDGDVGGPW-NH2 | 8 | 0.50 ± 0.03 |
| Ac-YTTNPRKLYDY-HN2 | 9 | 0.20 ± 0.02 |
| Ac-DVPQ-NH2 | 10 | >1,000 |
| Ac-RKLYDY-NH2 | 11 | 0.12 ± 0.02 |
| Ac-KLYDY-NH2 | 12 | 0.10 ± 0.03 |
| Ac-LYDY-NH2 | 13 | >1,000 |
| Biotin-PRKLYDY | 1 | 0.23 ± 0.02 |
| N-term-S-tag-Kringle 5 | — | 0.20 ± 0.05 |
| ¹²⁵IK5 (dog) | — | 0.50 ± 0.08 |

^{a}HMVECs were added to the bottom of a 48 well Boyden chamber and allow to attach to the membrane for 2 hours (25). The chambers were inverted and media containing 3 ng/ml VEGF and peptides was added to each well. The cells were incubated for 4 hours at 37° C. in 5% CO₂ to migrate. Finally, cells that did not migrate across the membranes were scraped off and the cells that migrated were stained and counted. IC50 values were determined by comparing
migrated cells in non-treated wells to treated wells. Each IC50 value listed is an average of triplicates.

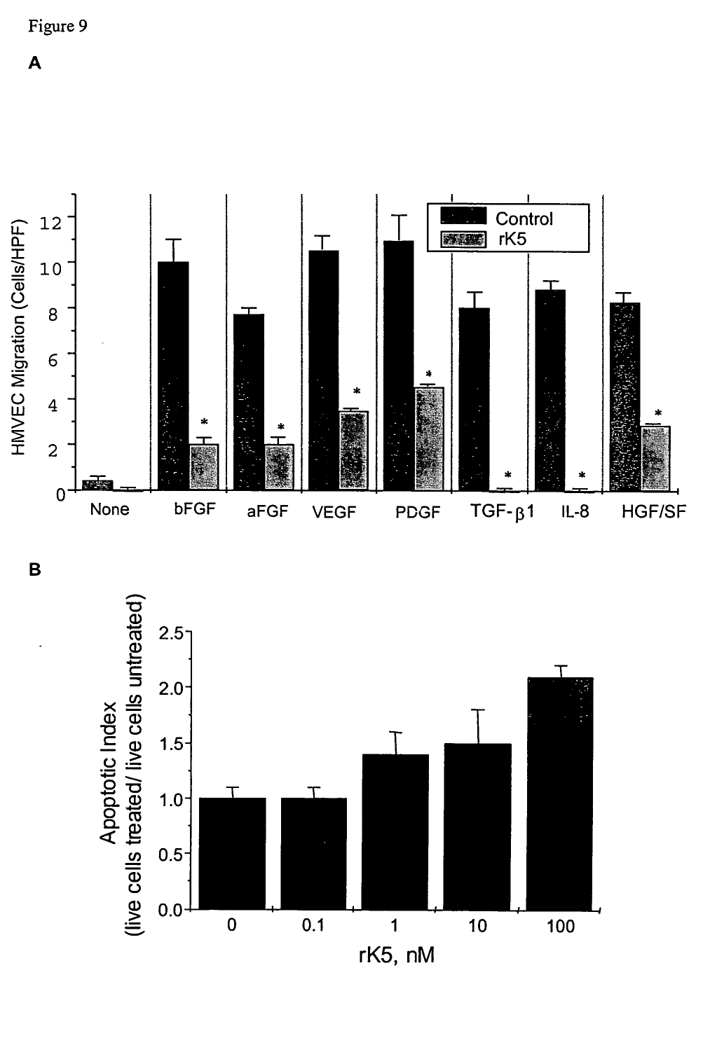

Recombinant K5 inhibited endothelial cell migration induced by a wide variety of inducers of angiogenesis including aFGF, bFGF, IL-8, PDGF, TGF-β and VEGF (FIG. 9A). It was selective for endothelial cells since it failed to inhibit the migration of neutrophils and fibroblasts even when tested at concentrations up to 1000-fold higher than that at which it inhibited endothelial cell migration (data not shown). Recombinant K5 also showed selectivity for inhibition of proliferation of stimulated endothelial cells, and did not cause inhibition of tumor or primary cell proliferation at concentrations as high as 100 μM (Table 2 below).

TABLE 2
Inhibition of tumor cell proliferation by rK5

IC50 values for cell proliferation^{a}

| Tumor | rK5 | Adriamycin |
|---|---|---|
| Cell Lines | (μg/ml) | (μg/ml) |
| HT-29 | >100 | 0.118 ± 0.008 |
| A549 | >100 | 0.051 ± 0.006 |
| LNCaP | >100 | 0.41 ± 0.10 |
| P388 | >100 | 0.0026 ± 0.0002 |
| B16F10 | >100 | 0.0032 ± 0.0004 |
| PC3 | >100 | NT^{b} |
| MDA 445 | >100 | NT |
| D54 | >100 | 0.005 ± 0.0004 |
| HT1080 | >100 | 0.002 ± 0.0003 |
| Lewis Lung | >100 | NT |
| Carcinoma | | |
| ^{a}Tumor cells were plated at 2000 cells per well in a 96 well plate in full media and allowed to attached overnight. rK5 or Adriamycin was added to the wells in fresh media and allowed to grow for 72 hours. The number of cells was | | |
| # measured by AQeous cell proliferation kit. IC50 values were determined by comparing treated cells with non-treated cells. Each test was run in quadruplicate. | | |

^{b}NT = not tested

Apoptosis of stimulated endothelial cells was induced by rK5 (FIG. 9B) in a dose dependent manner, indicating that the binding of rK5 initiates a cell-signaling cascade leading to cell death.

To further explore the binding of rK5 to endothelial cell surfaces, linear peptides made from various regions of K5 were also tested in the endothelial cell migration assay (Table 1, supra). Peptides from the lysine-binding site of K5 displayed the highest activity at blocking stimulated endothelial cell migration. Peptides PRKLYDY (SEQ ID NO:1), KLYDY (SEQ ID NO:12) and KLYD (SEQ ID NO:14) were equally potent compared to rK5 in the inhibition of migration of activated endothelial cells. However, peptides outside of this binding site pocket or tri-peptides from the active site pocket were inactive at blocking endothelial cell migration. An N-terminally biotinylated PRKLYDY (SEQ ID NO:1) peptide and a S-protein tagged rK5 were similarly active for the inhibition of stimulated endothelial cell migration. These probes were important for the isolation of the cell surface receptor for rK5.

### EXAMPLE 7

### GRP78 is an Endothelial Cell Surface rK5 Binding Protein

To reduce the cell surface non-specific protein binding that is often observed with proteins, an immobilized N-terminally biotinylated PRKLYDY (SEQ ID NO:1) peptide was used to isolate K5 binding proteins from endothelial cell surfaces. Bound proteins were then eluted with excess rK5. Mass spectrometric sequencing of tryptic peptides from the major protein band (˜80 kDa) revealed sequences corresponding to glucose-regulated protein 78 (GRP78)(78 kDa). That GRP78 is a cell surface binding protein for rK5 was further confirmed by co-precipitation of biotinylated surface proteins with S-tagged-K5. The major bound biotinylated proteins eluted with excess rK5 from immobilized S-tagged-K5 were identified by mass spectrometic analysis as GRP78 and GP96.

### EXAMPLE 8

### Characterization of GRP78 Binding to rK5

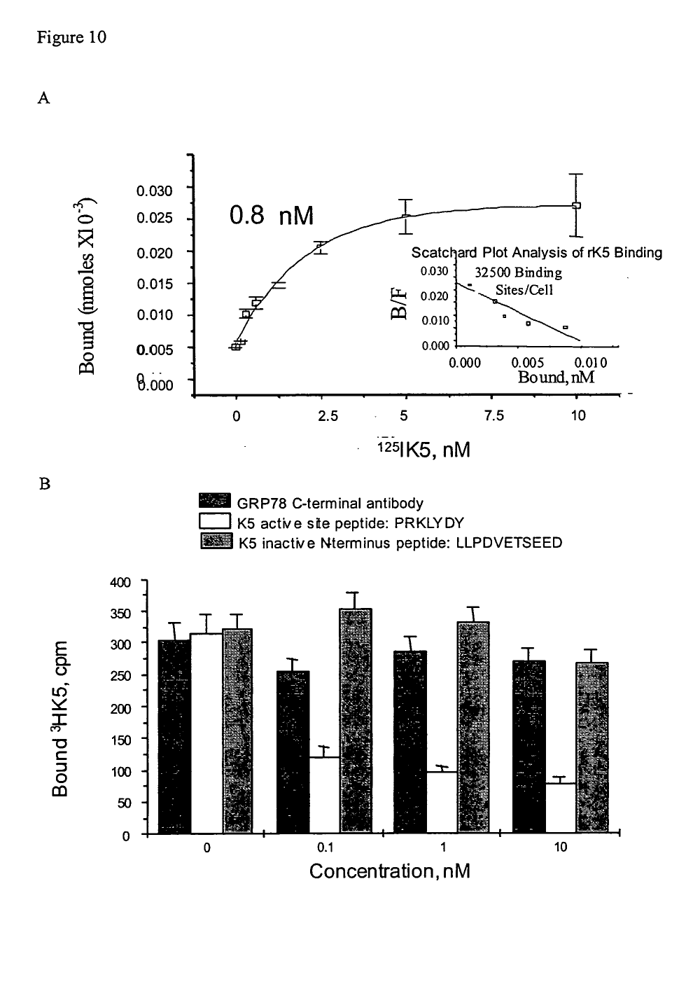

The possibility that rK5 binds to GRP78 on endothelial cells was further explored by the examination of the amount of surface expressed GRP78 compared to rK5 binding. Labeled K5 (¹²⁵IK5 (dog) or human ³HK5) bound specifically to these cells with a Kd of 0.8 nM (FIG. 10A), which is the same order of magnitude for rK5's inhibition of endothelial cell migration (0.25 nM) and induction of endothelial cell apoptosis (1 nM). A goat polyclonal antibody recognizing the N-terminal region of GRP78 inhibited the binding of ¹²⁵IKS to proliferating endothelial cells in a concentration-dependent manner (FIG. 1). In comparison, a goat polyclonal antibody raised against the C-terminal region of GRP78 had no effect on cellular ¹²⁵IK5 binding. Competition binding with the K5 active site peptide, PRKLYDY (SEQ ID NO:1, residues 80-86), but not the inactive rK5 N-terminal peptide, LLPDVETPSEED (SEQ ID NO:2, residues 1-12), blocked ³HK5 binding to endothelial cells in a concentration-dependent manner (FIG. 10B). These results demonstrate that GRP78 and rK5 associate on the surface of proliferating endothelial cells.

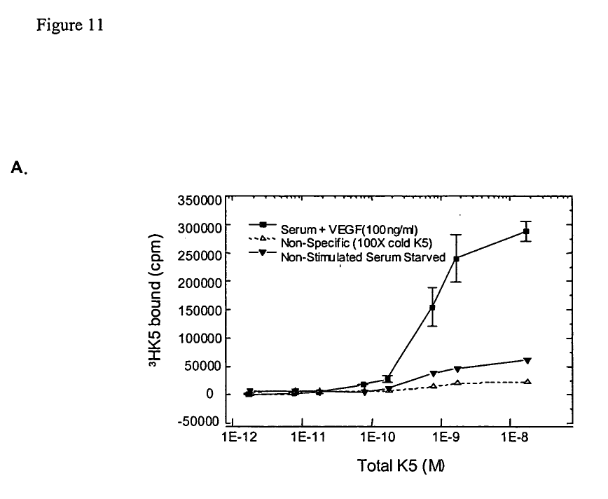

To investigate the specificity of GRP78/rK5 interaction, a mutant rK5(K82A) was used to compete with H³K5 binding to endothelial cell surfaces. Unlike rK5, the mutant rK5(K82A) at concentrations up to 500 nM did not inhibit the binding of ³HK5 or reduced the binding of a N-terminal GRP78 antibody on stimulated endothelial cell surfaces as determined by immunohistochemical(IHC) analysis. The binding of rK5 to endothelial cells is dramatically reduced in starved, quiescent cells compared to VEGF/bFGF stimulated cells (FIG. 11). Analysis of GRP78 cell surface expression, using immunohistochemical techniques, displayed a readily detectable increase in GRP78 expression on stimulated endothelial cell surfaces compared to cells after starvation. These results taken together indicate that the increase in rK5 binding to endothelial cell surfaces correlates with the amount of GRP78 expression on cells.

### EXAMPLE 9

### An N-Terminal Antibody to GRP78 or Reduced Expression of GRP78 Inhibits the Activity of rK5 on Endothelial Cells

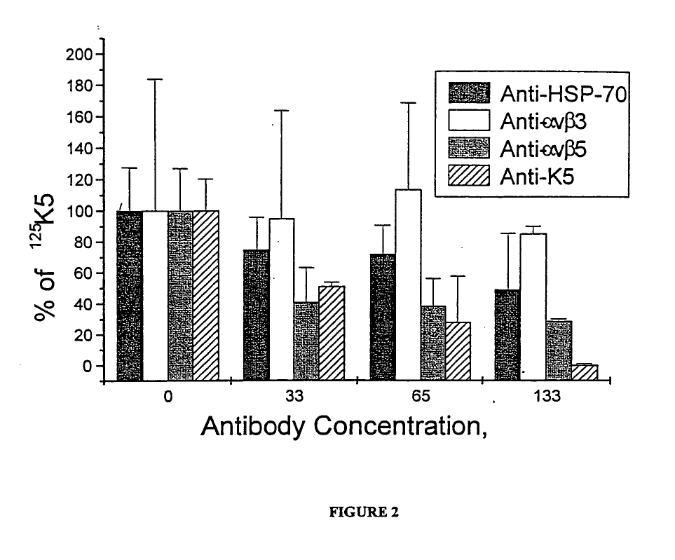

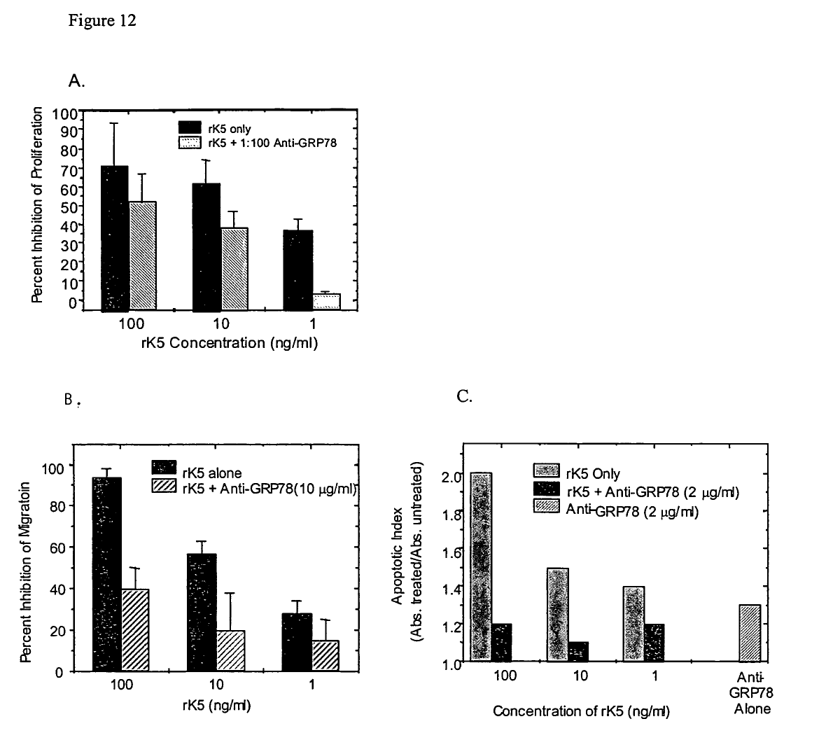

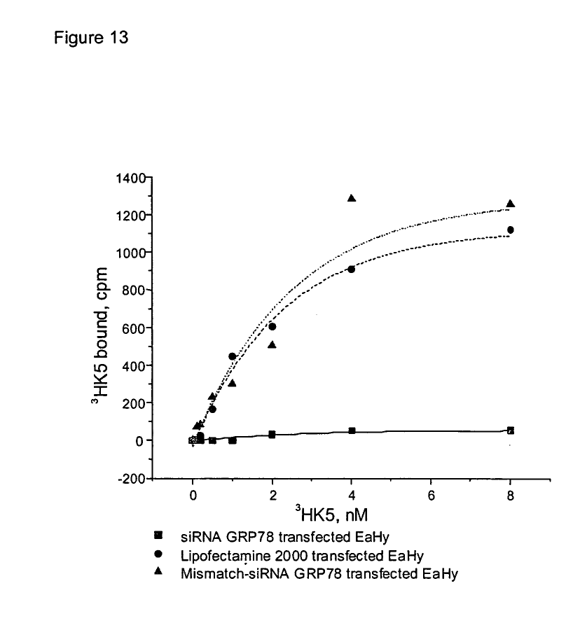

The previous results suggest GRP78 plays a role in the activity of rK5 on endothelial cells. An N-terminal GRP78 polyclonal antibody blocked the inhibition caused by rK5 on stimulated endothelial cell proliferation in a concentration-dependent manner (FIG. 12A), whereas antibodies to unrelated proteins, HSP70, fibrin and αVβ3, had no effect except at very high concentrations (FIG. 2). The same N-terminal GRP78 antibody also blocked the rK5-induced inhibition of endothelial cell migration (FIG. 12B) and rK5-induced apoptosis of endothelial cells (FIG. 12C). The expression of GRP78 on endothelial cells was significantly reduced by the transfection of a siRNA for GRP78 but not by the transfection of a scrambled siRNA. The binding of rK5 to GRP78 siRNA transfected HMVEC cells was very low compared to the binding of rK5 to scrambled siRNA transfected cells (FIG. 13). These results confirm the requirement for GRP78 expression for rK5 inhibitory activity on endothelial cells.

### EXAMPLE 10

### Direct Binding of rK5 with Recombinant GRP78

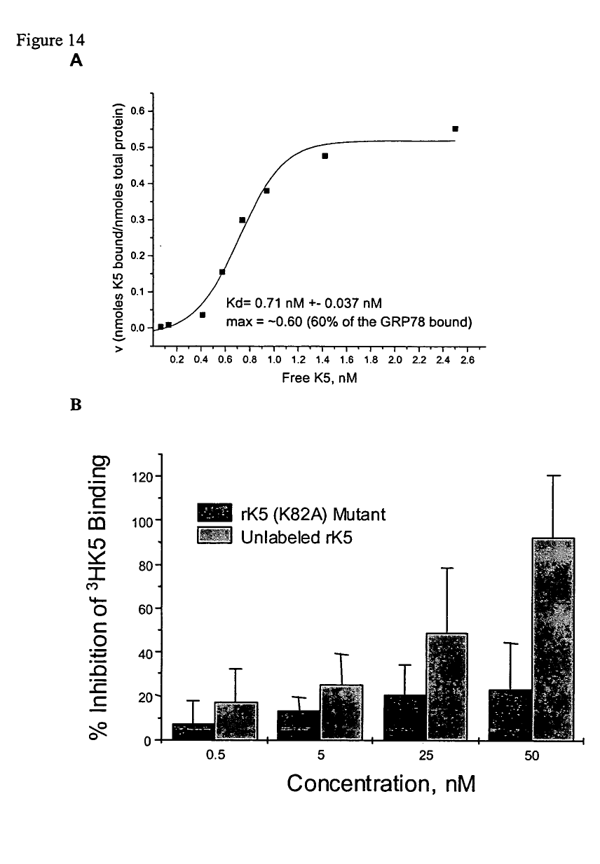

In order to directly observe the binding of GRP78 and rK5, yeast-expressed recombinant human GRP78 (rGRP78) protein and ³HK5 were used. Equilibrium dialysis of rGRP78 and ³HK5 revealed direct binding of rGRP78 and rK5 in a concentration dependent manner with a Kd of approximately 0.7 nM (FIG. 14A). This binding could be blocked by the addition of unlabeled rK5 (FIG. 14B). However, the rK5(K82A) mutant in concentrations up to 25-fold excess of GRP78 concentration did not block binding, confirming that GRP78 binds with high affinity and specificity to rK5.

### EXAMPLE 11

### Recombinant K5 Causes Apoptosis of Hypoxic Tumor Cells

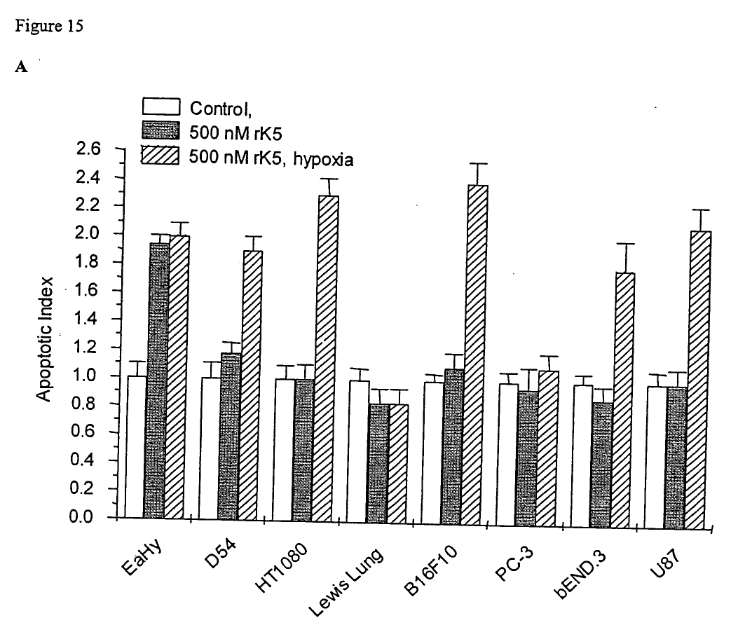

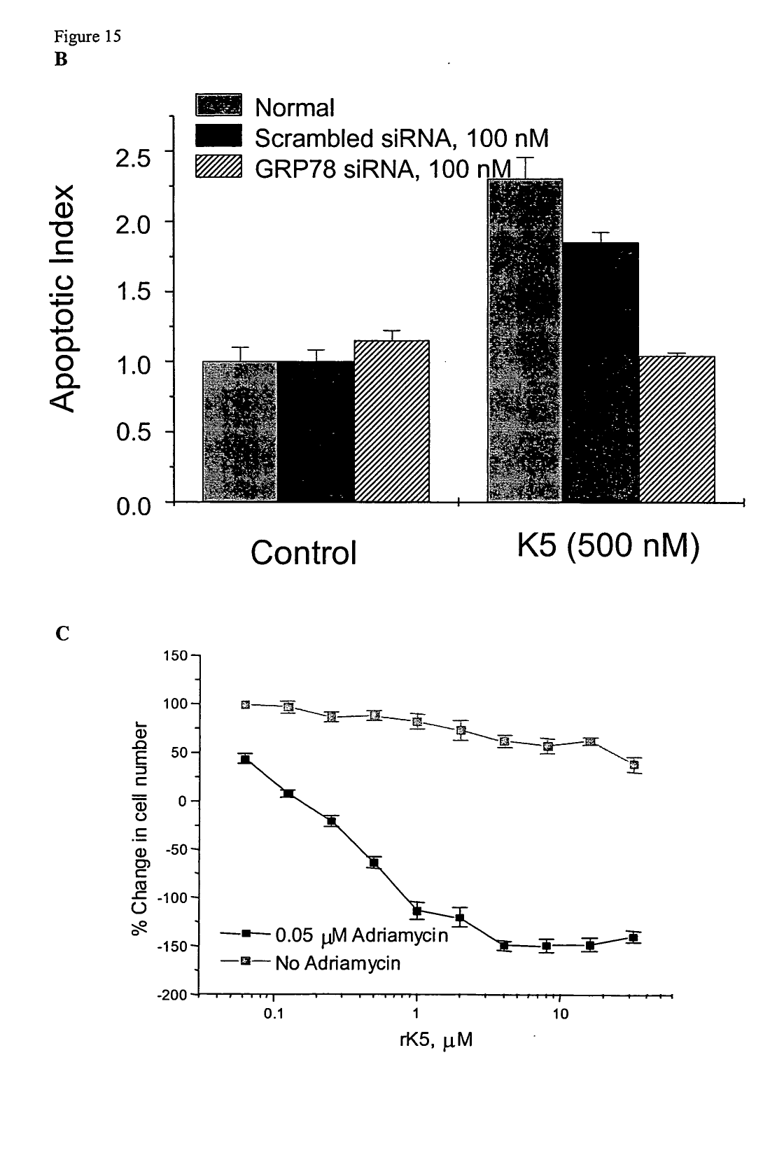

The results shown above demonstrate that the inhibitory activity of rK5 on endothelial cells is mediated through cell surface GRP78. These results also show that K5 does not affect tumor cell proliferation in normal conditions in vitro (Table 2 supra). However, published data indicate that tumor cells up-regulate surface GRP78 expression under stressed conditions (Koomagi R, Mattern J, Volm M. Glucose-related protein (GRP78) and its relationship to the drug-resistance proteins P170, GST-pi, LRP56 and angiogenesis in non-small cell lung carcinomas. Anticancer Res. 1999 Sep-Oct; 19(5B):4333-6). When hypoxia and/or the addition of cytotoxic stress are applied to tumor cells, an apoptosis-resistant phenotype often emerges. This resistance has been linked to GRP78 surface expression (Koomagi et al., supra). Therefore, the effect of rK5 on tumor cells under stressed hypoxic conditions was examined. Hypoxia increased GRP78 expression on HT1080 human fibrosarcoma cells at least 4 fold. When rK5 was added to these cells under hypoxic conditions (5% O₂, 95% N2) there was a greater than two-fold increase in apoptosis within 24 hours (FIG. 15A) while hypoxia alone or the addition of rK5 without hypoxia had no significant effect on cellular apoptosis. To determine if the pro-apoptotic effect of rK5 on hypoxic tumor cells was specific to HT1080 cells, we tested apoptosis rates for various tumor cell lines exposed to rK5 under hypoxia. As seen in FIG. 15B, rK5 induced apoptosis in 8 out of 10 tumor cell lines tested under hypoxic conditions, the two exceptions being Lewis Lung and PC-3 cell lines. To better understand this phenomenon, GRP78 surface expression was examined on PC-3 cells under hypoxic conditions. IHC analysis of PC-3 and Lewis Lung cells showed no increase in GRP78 expression under hypoxia (data not shown), which is in sharp contrast with the stress induced GRP78 expression on HT1080 cells. To make the expression of GRP78 on HT1080 cells appear more like PC-3 cells, an siRNA to GRP78 was used to knock down GRP78 protein expression. Much like the siRNA knock down of GRP78 expression in HMVECs, there was approximately a 90% decrease in GRP78 expression in transfected HT1080 (GRP78⁻) cells (data not shown). This decrease in GRP78 protein expression significantly eliminated the activity of rK5 on hypoxia-stressed, transfected HT1080 cells (FIG. 15C). However, rK5 induced apoptosis of the hypoxia-stressed, scrambled siRNA transfected HT1080 cells, similar to the control non-transfected cells. This data shows the necessary of GRP78 expression for rK5 induction of apoptosis on tumor cells.

In addition to hypoxia, reports indicate that cytotoxic agents can cause up regulation of GRP78 resulting in tumor cell resistance to cell growth inhibition (Koomagi et al., supra). Adriamycin, a cytotoxic agent, was tested for its ability to sensitize D54 glioma cells to growth inhibition by rK5. A concentration of Adriamycin (50 nM) that did not inhibit D54 cell growth was very effective against cell growth in combination with rK5 (FIG. 15D).

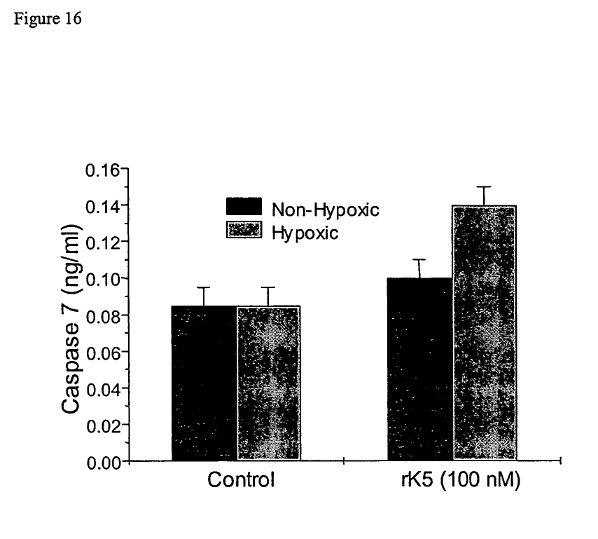

Recent reports have shown that the ATPase domain of GRP78 binds to procaspase 7, blocking its activation and decreasing stress induced cellular apoptosis. NMR studies with a recombinant GRP78 ATPase domain show direct binding of rK5 with the ATPase domain of GRP78. We have also shown that the GRP78 ATPase domain on stressed tumor cells is extracellular. If rK5 is internalized and competes with procaspase 7 for GRP78 binding, then stressed tumor cells treated with rK5 should show an increase in caspase 7 activity compared to untreated cells. OAs FIG. 16 shows that a significant increase in caspase 7 activity occurred in HT1080 cells when treated with rK5 and hypoxia, consistent with this hypothesis.

1. A method of identifying a composition which inhibits activation of an endothelial cell receptor comprising the steps of:
a) constructing a vector comprising a nucleotide sequence encoding said endothelial cell receptor and a nucleotide sequence encoding a reporter molecule, said nucleotide sequence encoding said reporter molecule being operably linked to said nucleotide sequence encoding said endothelial cell receptor;
b) introducing said vector into a host cell for a time and under conditions suitable for expression of said endothelial cell receptor;
c) exposing said host cell to a composition which may inhibit activation of said endothelial cell receptor and a substrate specific for said reporter molecule; and
d) measuring the signal generated by reaction of said reporter molecule and said substrate in comparison to that produced by a control host cell, a smaller signal by said host cell of (c) indicating that said composition will inhibit activation of said endothelial cell receptor.

2. The method of claim 1, wherein said endothelial cell receptor is GRP78.

3. The method of claim 2, wherein said composition is kringle 5 (K5).

4. A method of identifying a composition which inhibits expression of an endothelial cell receptor comprising the steps of:
a) adding an antibody selected from the group consisting of a monoclonal antibody and a polyclonal antibody produced against said endothelial cell receptor to a solid phase;
b) adding known concentrations of said endothelial cell receptor, exposed to said composition, to said solid phase, in order to form a first complex between said antibody and said known concentrations of said endothelial cell receptor;
c) adding a second antibody to said first complex, selected from the group consisting of a monoclonal antibody and a polyclonal antibody produced against said endothelial cell receptor for a time and under conditions sufficient for formation of a second complex between said first complex and said second antibody;
d) contacting said second complex with an indicator reagent which comprises a signal-generating compound attached to an antibody against said antibody of said second complex, for a time and under conditions sufficient for formation of a third complex; and
e) detecting the presence of a measurable signal, absence of said signal indicating said composition inhibits expression of said endothelial cell receptor and presence of said signal indicating said composition does not inhibit expression of said endothelial cell receptor.

5. The method of claim 4 wherein said endothelial cell receptor is GRP78.

6. The method of claim 5 wherein a composition which inhibits expression of said endothelial cell receptor is K5.

7. A method of identifying a composition which binds to the GRP78 receptor comprising the steps of:
a) exposing said receptor to said composition for a time and under conditions sufficient for formation of a complex; and
b) determining presence or absence of said complex, presence of said complex indicating a composition which binds to said receptor.

8. The method of claim 7 wherein said compound is attached to an indicator molecule capable of generating a detectable signal.

9. The method of claim 7 wherein said compound which binds to said GRP78 receptor is K7 or a functional equivalent thereof.

10. A method of preventing or treating angiogenesis in a patient in need of said prevention or treatment comprising the step of administering to said patient in an amount of a composition which binds to at least one endothelial cell receptor sufficient to effect said prevention or treatment.

11. The method of claim 10 wherein said endothelial cell receptor is GRP78.

12. The method of claim 10 wherein said composition is K5.

13. A method of inhibiting tumor cell growth in a patient in need thereof, comprising administering to said patient an amount of a composition which binds to GRP78 on a tumor cell sufficient to effect said inhibition.

14. The method of claim 13 wherein said composition is K5.

15. The method of claim 13 wherein said tumor cell is hypoxic.

16. A method of inducing apoptosis in a tumor cell comprising administering to said tumor cell an amount of a composition which binds to GRP78 on said tumor cell sufficient to effect said induction.

17. The method of claim 16 wherein said composition is K5.

18. The method of claim 16 wherein said tumor cell is hypoxic.
